(19) United States
(12) Patent Application Publication
(10) Pub. No.: US 2017/0143919 A1
(43) Pub. Date: May 25, 2017

(54) CUFF AND TRACHEAL TUBE

(71) Applicant: Anri FUJII, Kanagawa (JP)

(72) Inventor: Anri FUJII, Kanagawa (JP)

(73) Assignee: TERUMO KABUSHIKI KAISHA, TOKYO (JP)

(21) Appl. No.: 15/331,305

(22) Filed: Oct. 21, 2016

(30) Foreign Application Priority Data
Nov. 19, 2015 (JP) 2015-226593

(51) Int. Cl. A61M 16/04

(52) U.S. Cl. CPC A61M 16/0445; A61M 16/0443; A61M 16/0486; A61M 16/0463; A61M 16/044

(57) ABSTRACT

Provided is a cuff having a trunk portion capable of deflation and inflation. The trunk portion in a state of the inflation is rotationally symmetric with respect to a prescribed axis and has a tapered part whose diameter gradually increases centering at the prescribed axis as it goes from one end side towards the other end side of the prescribed axis. An angle made by the prescribed axis and the tapered part is greater than or equal to 10° and less than or equal to 12°. A wrinkle area ratio as a ratio between a wrinkle area of a part of the trunk portion where wrinkles form and a contact area of a part of the trunk portion contacting an inner wall of a trachea in a state in which the trunk portion is inflated in the trachea is less than or equal to 4.4%.

### CROSS REFERENCE TO RELATED APPLICATIONS

This application is based on and claims priority to Japanese Application No. JP2015-226593, filed on Nov. 19, 2015, the entire contents of which are herein incorporated herein by reference.

### BACKGROUND

The present disclosure relates to a cuff and a tracheal tube.

As a related art, tracheotomy tubes are well known as tracheal tubes used for directly connecting the inside of the trachea of a patient having difficulty with spontaneous respiration to the outside of the patient's body and thereby facilitating respiration. A cuff capable of deflation and inflation is attached on the outer peripheral surface of a tube main body of the tracheotomy tube. The tracheotomy tube with the cuff in a deflated state is inserted into the inside of the patient's trachea and thereafter the cuff is inflated, by which the cuff is brought into contact with the inner wall of the trachea and the tracheotomy tube is indwelled in the trachea (see Japanese Patent No. 5271898 and Japanese Patent Laid-open No. 2000-167060, for example).

### SUMMARY

However, when a tracheal tube is used in the patient's trachea with the cuff in the inflated state, foreign matter such as phlegm, saliva, blood and accidentally-swallowed object accumulates on the upstream side (the side of a jaw) of the trachea. The accumulated foreign matter can be removed by suction through a lumen formed in the tracheal tube, for example.

Nevertheless, when the cuff is inflated in the trachea, there are cases where wrinkles form on the outer surface of the cuff and a gap due to the wrinkles appears between the outer surface of the cuff and the inner wall of the patient's trachea. When such a gap due to wrinkles appears, foreign matter enters the gap and the removal of foreign matter by means of suction becomes difficult.

The object of the present disclosure, which has been made in consideration of the aforesaid problem, is to provide a cuff and a tracheal tube capable of improving the foreign matter suction performance.

A cuff as a first mode of the present disclosure is a cuff having a trunk portion capable of deflation and inflation. The trunk portion in a state of the inflation is rotationally symmetric with respect to a prescribed axis and has a tapered part whose diameter gradually increases centering at the prescribed axis as it goes from one end side towards the other end side of the prescribed axis. An angle made by the prescribed axis and the tapered part is greater than or equal to 10° and less than or equal to 12°. A wrinkle area ratio as a ratio between a wrinkle area of a part of the trunk portion where wrinkles form and a contact area of a part of the trunk portion contacting an inner wall of a trachea in a state in which the trunk portion is inflated in the trachea is less than or equal to 4.4%.

As an embodiment of the present disclosure, a film thickness of the trunk portion is desired to be greater than or equal to 0.06 mm and less than 0.15 mm.

As an embodiment of the present disclosure, the trunk portion is desired to be formed with a material having a Young's modulus greater than or equal to 0.32 MPa and less than or equal to 0.39 MPa.

A tracheal tube as a second mode of the present disclosure is a tracheal tube having a tube main body equipped with any one of the aforesaid cuffs. The prescribed axis is a central axis line of an outer peripheral surface of the tube main body. The one end side is a distal side of the tube main body.

According to the present disclosure, a cuff and a tracheal tube capable of improving the foreign matter suction performance can be provided.

### BRIEF DESCRIPTION OF THE DRAWINGS

FIG. 1 is a diagram showing a state in which a tracheal tube according to an embodiment of the present disclosure has been indwelled in a trachea;

FIG. 2 is a perspective view individually showing a tube main body of the tracheal tube in FIG. 1;

FIG. 3 is a lateral view showing the general features of a cuff of the tracheal tube in FIG. 1;

FIG. 4 is a diagram showing an example of a contact region recorded on a laminate film obtained in an experiment conducted by the present inventors;

FIGS. 5A to 5C are diagrams showing three types of the shape of the cuff used in experiments by the present inventors;

FIG. 6 is a chart showing characteristics of the cuffs of eight types of tracheal tubes used in the experiments by the present inventors;

FIG. 7 is a diagram schematically showing a wrinkle end formed on a cuff of a tapered type;

FIG. 8 is a diagram showing an example of a cross section of a cuff with a welding portion welded in a state of extending from a trunk portion towards a distal side and another welding portion welded in a state of being folded back towards the trunk portion; and

FIG. 9 is a flow chart showing an example of a method for attaching the cuff to the tracheal tube.

### DETAILED DESCRIPTION OF THE PREFERRED EMBODIMENT

Referring now to the drawings, a description will be given in detail of a preferred embodiment of a tracheal tube in accordance with the present disclosure. Note that each member appearing in multiple drawings is assigned the same reference symbol throughout the drawings.

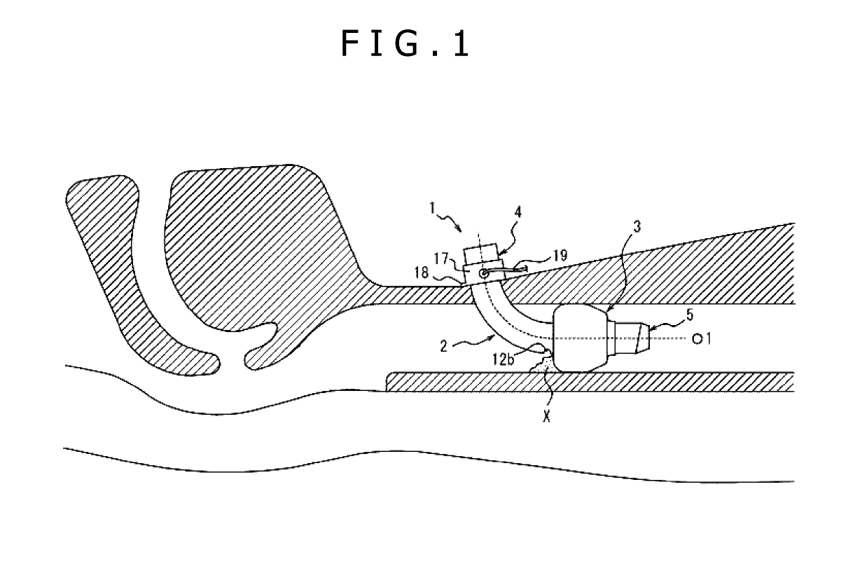

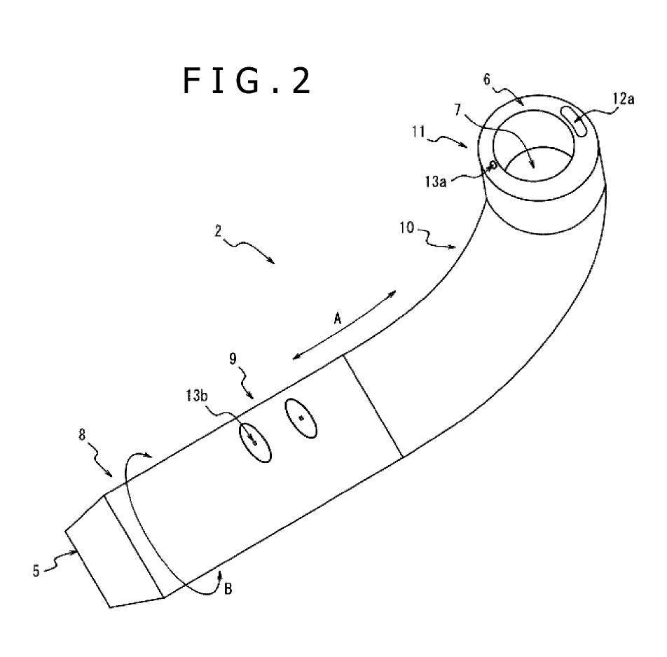

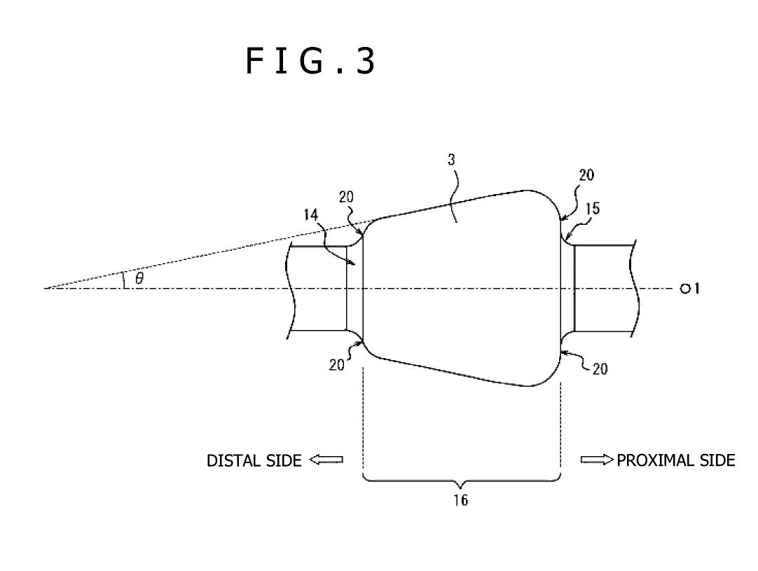

FIG. 1 is a diagram showing a state in which a tracheal tube 1 according to an embodiment of the present disclosure has been indwelled in the trachea. FIG. 2 is a perspective view individually showing a tube main body 2 of the tracheal tube 1 in FIG. 1. FIG. 3 is a lateral view showing the general features of a cuff 3 of the tracheal tube 1 in FIG. 1.

As shown in FIG. 1, the tracheal tube 1 includes the tube main body 2, the cuff 3 and a flange member 4. The cuff 3, capable of deflation and inflation, is attached on an outer peripheral surface of the tube main body 2. The flange member 4 is attached to one end portion of the tube main body 2.

The tube main body 2 demarcates a hollow portion 7 that penetrates the tube main body 2 from a distal end 5 to a proximal end 6 in the extending direction of a central axis line O1 of the outer peripheral surface of the tube main body 2 (hereinafter referred to simply as a “central axis line direction A”). In the state in which the tracheal tube 1 has been inserted from the outside and indwelled in the trachea, the airway for respiration is secured by the hollow portion 7. Note that the distal end 5 of the tube main body 2 denotes the distal end of the tube main body 2, that is, one end situated on the side of the tracheal bifurcation in the state in which the tracheal tube 1 has been indwelled in the trachea. The proximal end 6 denotes the proximal end of the tube main body 2, that is, the other end situated on the side of the jaw in the state in which the tracheal tube 1 has been indwelled in the trachea.

The tube main body 2 has a distal portion 8 which includes the distal end 5, a cuff attachment portion 9 which connects to the distal portion 8 on the side of the proximal end 6 in the central axis line direction A and to whose outer peripheral surface the cuff 3 is attached, a curved portion 10 which connects to the cuff attachment portion 9 on the side of the proximal end 6, and a proximal portion 11 which connects to the curved portion 10 on the side of the proximal end 6 and includes the proximal end 6. Put another way, the distal portion 8 of the tube main body 2 is connected to the proximal portion 11 via the cuff attachment portion 9 and the curved portion 10. Note that the flange member 4 is attached to the proximal portion 11.

Two hollow portions extending along the central axis line O1 are demarcated in the wall of the tube main body 2 between the outer peripheral surface of the tube main body 2 and the inner peripheral surface of the tube main body 2, which demarcates the hollow portion 7. Specifically, the tube main body 2 includes a first lumen and a second lumen formed in the wall and extending along the central axis line O1 from a first proximal opening 12a and a second proximal opening 13a demarcated on a proximal surface. Note that, while the small-diameter first and second lumens demarcated in the wall are also hollow portions, the first and second lumens are referred to as “lumens” here for convenience of the explanation, that is, in order to discriminate them from the large-diameter hollow portion 7 for securing the airway.

The first lumen extends from the first proximal opening 12a of the proximal surface to a prescribed position on the side of the proximal portion 11 with respect to the cuff attachment portion 9 and the cuff 3. The first lumen connects to the outside of the tube main body 2 via a suction hole portion 12b formed at the prescribed position to reach and penetrate the outer peripheral surface of the tube main body 2. Note that the suction hole portion 12b in this embodiment is a suction hole formed in the curved portion 10 as the position on the side of the proximal portion 11 with respect to the cuff attachment portion 9 and the cuff 3. The first lumen sucks in and removes foreign matter X such as phlegm, saliva, blood and accidentally-swallowed object that has accumulated on the upstream side (the side of a jaw) with respect to the cuff 3 indwelled in the trachea.

The second lumen extends from the second proximal opening 13a of the proximal surface to the position of the cuff attachment portion 9 and the cuff 3. The second lumen connects to the outside via a channel 13b formed at the position to reach and penetrate the outer peripheral surface of the tube main body 2. Therefore, the cuff 3 can be inflated by supplying fluid such as air from the second proximal opening 13a of the second lumen to the inside of a space demarcated by the outer peripheral surface of the cuff attachment portion 9 and the inner surface of the cuff 3 (annular space) via the channel 13b by using a syringe or the like. Further, the cuff 3 in the inflated state can be deflated by sucking in the fluid from the annular space via the channel 13b and the second proximal opening 13a of the second lumen. As explained above, the second lumen is a lumen used for deflating and inflating the cuff 3. The second lumen will hereinafter be referred to as a “cuff lumen.”

As the constituent material of the tube main body 2, various types of resins such as silicone, polyvinyl chloride, polyethylene, polypropylene, cyclic polyolefin, polystyrene, poly-(4-methyl-pentene-1), polycarbonate, acrylic resin, acrylonitrile-butadiene-styrene copolymer, polyester (e.g., polyethylene terephthalate), butadiene-styrene copolymer and polyamide (e.g., nylon 6, nylon 6/6, nylon 6/10 and nylon 12) are usable, for example. Above all, using resins like polyvinyl chloride, polypropylene, cyclic polyolefin, polyester and poly-(4-methyl-pentene-1) is desirable in terms of easiness of shaping.

The flange member 4 is attached to the proximal portion 11 (see FIG. 2) of the tube main body 2 as shown in FIG. 1. When the tracheal tube 1 is indwelled in the patient's body by inserting the tube main body 2 from the outside of the patient's body into the patient's trachea, the flange member 4 contacts the patient's skin and thereby fixes the distal portion 8 at an appropriate position in the trachea. Into the flange member 4, the proximal portion 11 of the tube main body 2 is inserted. The flange member 4 includes a tube portion 17 in a cylindrical shape and a flange portion 18 in a tabular shape. The tube portion 17 is attached to the tube main body 2 by fitting the tube portion 17 onto the tube main body 2. The flange portion 18 protrudes outward in the radial direction from the outer wall of the tube portion 17. The flange portion 18 is attached to the patient's skin in the state in which the tracheal tube 1 has been indwelled.

The first lumen connects to the outside of the tracheal tube 1 on the proximal side of the tracheal tube 1 via a corresponding communication hole formed through the tube portion 17. The suction of foreign matter X such as accidentally-swallowed object is carried out by connecting a syringe, suction pump or the like to an end of a suction tube 19, whose other end has been engaged with the communication hole of the tube portion 17 exposed to the outside of the patient's body, and operating the syringe, suction pump or the like for the suction.

The cuff lumen connects to the outside of the tracheal tube 1 on the proximal side of the tracheal tube 1 via a corresponding communication hole formed through the tube portion 17. Therefore, by connecting a syringe or the like to an end of a cuff tube, whose other end has been engaged with the communication hole of the tube portion 17 exposed to the outside of the patient's body, and operating the syringe or the like outside the patient's body, fluid can be supplied to and sucked from the annular space of the cuff 3, by which the inflation and deflation of the cuff 3 can be carried out.

The flange member 4 can be formed of a material similar to that of the tube main body 2, for example.

The cuff 3 is attached on the outer peripheral surface of the cuff attachment portion 9 of the tube main body 2. The cuff 3 can be inflated by the pressure of the fluid supplied to the annular space demarcated by the outer peripheral surface of the cuff attachment portion 9 and the inner surface of the cuff 3 via the aforementioned cuff lumen of the tube main body 2. Thus, when the tracheal tube 1 is inserted into the trachea from the outside and the tracheal tube 1 is indwelled at a prescribed position, the cuff 3 is inflated by supplying the fluid to the annular space via the cuff lumen of the tube main body 2. Accordingly, the outer surface of the inflated cuff 3 makes close contact with the inner wall of the trachea, the cuff 3 is held on the inner peripheral wall surface of the cuff 3 due to factors such as frictional force between the outer surface of the cuff 3 and the inner wall of the trachea, and the cuff 3 occludes the trachea around the tube main body 2. Consequently, the position of the cuff 3 in the trachea is fixed and the tracheal tube 1 can be indwelled at the aforementioned prescribed position.

In cases like extracting and removing the tracheal tube 1 from the trachea to the outside or adjusting the position where the tracheal tube 1 is indwelled, the cuff 3 is deflated by sucking the fluid from the annular space by use of the cuff lumen. By this operation, the tube main body 2 of the tracheal tube 1 can be moved inside and along the trachea.

End portions of the cuff 3 on the side of the proximal end 6 and the side of the distal end 5 in the central axis line direction A are joined to the outer peripheral surface of the cuff attachment portion 9 by welding, ultrasonic welding or the like in the entire region in a circumferential direction B (see FIG. 2) of the tube main body 2. Accordingly, the aforementioned annular space is formed by the inner surface of the cuff 3 and the outer peripheral surface of the cuff attachment portion 9.

The cuff 3 will be explained in more detail below by referring to FIG. 3. In FIG. 3, only the cuff 3 attached to the tube main body 2 and parts of the tube main body 2 in the vicinity of the cuff 3 are shown. FIG. 3 illustrates a state in which the cuff 3 has been inflated to the maximum size. In other words, the cuff 3 takes on the state shown in FIG. 3 when the cuff 3 is inflated without being inserted into the trachea.

The cuff 3 has welding portions 14 and 15 on the side of the distal end 5 and the side of the proximal end 6 as portions welded to the tube main body 2. In the cuff 3, the external form of a trunk portion 16 between the welding portion 14 on the side of the distal end 5 and the welding portion 15 on the side of the proximal end 6 is like a circular truncated cone having the welding portion 14 on its upper base side, the welding portion 15 on its lower base side, and the central axis line O1 as its central axis. Thus, the trunk portion 16 is rotationally symmetric with respect to the central axis line O1 and has a tapered shape in the lateral view, with the diameter of the outer surface around the central axis line O1 increasing as it goes from the side of the distal end 5 towards the side of the proximal end 6. Both ends of the trunk portion 16, that is, portions of the trunk portion 16 connecting to the welding portions 14 and 15, are formed as gently curved surfaces. Note that boundaries between the trunk portion 16 and the welding portions 14 and 15 are assumed to be situated at inflection points 20 of the curves of the connecting portions of the trunk portion 16 in the lateral view of the cuff 3. The angle θ of the tapered part of the cuff 3 with respect to the central axis line O1 (taper angle) in the lateral view is 12°.

In the trunk portion 16, the length along the central axis line O1 (corresponding to the height of the circular truncated cone), that is, the distance between the welding portions 14 and 15 (cuff width), can be set appropriately. In this embodiment, the cuff width is 26 mm. Further, the maximum diameter of a circle formed by the outer surface of the trunk portion 16 on a plane orthogonal to the central axis line O1 (cuff diameter) can be set appropriately to suit the size of the patient's trachea, for example. In this embodiment, the maximum diameter is 26 mm, for example. The maximum diameter is desired to be at a size that allows the tracheal tube 1 to be indwelled in the trachea due to prescribed appropriate pressure applied from the outer surface of the cuff 3 to the inner wall of the patient's trachea when the cuff 3 is inflated.

The material of the film constituting the outer surface of the cuff 3 in this embodiment is flexible polyvinyl chloride having a Young's modulus of 0.34 MPa. As the material of the film constituting the outer surface of the cuff 3, it is also possible to use, for example, polyurethane, polyethylene, polypropylene, polyester, ethylene-vinyl acetate copolymer (EVA), silicone, or a material having flexibility made by mixing any of these materials. The film constituting the outer surface of the cuff 3 in this embodiment has a film thickness of 0.06 mm.

With the cuff 3 according to this embodiment satisfying the aforementioned conditions and the tracheal tube 1 equipped with the cuff 3, the formation of wrinkles on the outer surface of the cuff 3 when the cuff 3 is inflated in the trachea can be inhibited, and thus a gap due to the wrinkles hardly appears between the outer surface of the cuff 3 and the inner wall of the patient's trachea. Since a gap allowing in foreign matter such as accidentally-swallowed object hardly appears between the outer surface of the cuff 3 and the inner wall of the patient's trachea, the amount of foreign matter escaping the suction by use of the first lumen decreases. Therefore, the foreign matter suction performance can be improved by the cuff 3 and the tracheal tube 1 equipped with the cuff 3.

The present inventors conducted experiments and found that the aforementioned effects can be achieved by the cuff 3 according to this embodiment satisfying the aforementioned conditions and the tracheal tube 1 equipped with the cuff 3. The experiments conducted by the present inventors will be explained in detail below.

The present inventors conducted the experiments while paying attention to three elements: the film thickness, the shape and the material of the cuff. The present inventors carried out two types of experiments: a first experiment and a second experiment.

In the first experiment, by using tracheal tubes equipped with various types of cuffs differing in the three elements (the film thickness, the shape and the material of the cuff) and syringes simulating a human trachea, the state in which the tracheal tube has been indwelled in the trachea was simulated and the ratio between the area of a part where wrinkles have formed (wrinkle area) and the area of a part where the outer surface of the cuff and the syringe simulating the trachea are in contact with each other (contact area) was measured. The ratio of the wrinkle area to the contact area will hereinafter be referred to also as a wrinkle area ratio.

The concrete method of the first experiment is as follows: First, a laminate film is rounded into a cylindrical shape and inserted into a syringe (inner diameter: 20 mm) simulating the trachea so that the laminate film adheres to the inner wall surface of the syringe. As the syringe, a Terumo Syringe produced by Terumo Corporation was used. As the laminate film, a laminator film (thickness: 0.1 mm) produced by ASKA CORPORATION was used. Subsequently, a tracheal tube with a cuff in the deflated state having paint applied on its outer surface is inserted into the syringe, and the cuff is inflated with a constant pressure (e.g., 16 mmHg to 25 mmHg), by which the state in which the tracheal tube is indwelled in the trachea is simulated. In this case, the paint applied on the outer surface of the cuff sticks to a part of the laminate film (adhering to the inner wall surface of the syringe) in contact with outer surface of the cuff. In contrast, parts of the outer surface of the cuff where wrinkles have formed do not contact the laminate film, and thus the paint does not stick to the wrinkled parts. Then, the tracheal tube is removed from the syringe by deflating the cuff. Thereafter, the laminate film is peeled off from the inner wall surface of the syringe. On the laminate film peeled from the syringe, the wrinkle area ratio is measured based on a region to which the paint has stuck (contact region).

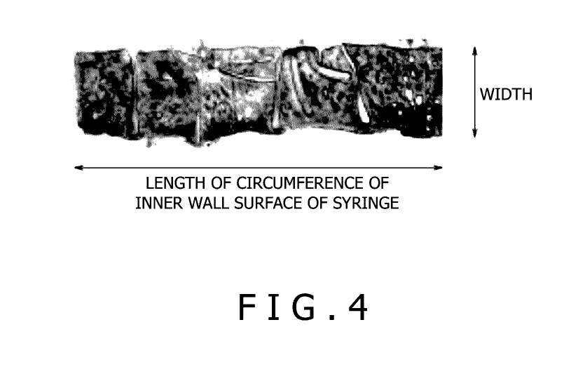

The method for measuring the wrinkle area ratio will be explained below by referring to FIG. 4. First, the contact area is calculated based on the contact region on the laminate film peeled from the syringe. The contact area is calculated as, for example, the product of the length of the circumference of the inner wall surface of the syringe and the width of the contact region in the direction orthogonal to the circumferential direction. In cases where the width of the contact region is not constant throughout the entire contact region, the average of the width across the entire contact region may be used, for example. Subsequently, the area of the parts in the contact region to which the paint has not stuck, that is, the area of the wrinkled parts (wrinkle area) is calculated. The wrinkle area is calculated by, for example, calculating the product of a wrinkle width and a wrinkle length in regard to each wrinkle and adding up the calculated products for all the wrinkled parts. Finally, the wrinkle area ratio is calculated by dividing the wrinkle area by the contact area and representing the quotient in percentage. Note that the expression “a wrinkle area ratio as a ratio between a wrinkle area of a part of the trunk portion where wrinkles form and a contact area of a part of the trunk portion contacting an inner wall of a trachea in a state in which the trunk portion is inflated in the trachea” in the present application means the “wrinkle area ratio” calculated by the experiment explained here.

In the second experiment, by using tracheal tubes equipped with various types of cuffs differing in the three elements (the film thickness, the shape and the material of the cuff) and syringes simulating a human trachea, the state in which the tracheal tube has been indwelled in the trachea was simulated. Liquid was poured into the syringe in a tilted state from above the cuff. The ratio between the amount of the liquid that flowed out from the bottom side of the cuff and the amount of the liquid poured from above the cuff was measured. The ratio of the amount of the liquid that flowed out from the bottom side of the cuff to the amount of the liquid poured from above the cuff will hereinafter be referred to also as a “trickle ratio.”

The concrete method of the second experiment is as follows: First, a tracheal tube with a cuff in the deflated state is inserted into the syringe, and the cuff is inflated with a constant pressure, by which the state in which the tracheal tube is indwelled in the trachea is simulated. Then, the syringe is tilted from the horizontal position by 30° so that the proximal side of the inserted tracheal tube is positioned above the distal side, by which a state in which the patient with the tracheal tube indwelled in the trachea is lying on the bed is simulated. In this state, a prescribed amount of liquid is poured into the syringe from above the cuff, that is, from the proximal side. The present inventors used RO (Reverse Osmosis) water in the second experiment. Three minutes after the pouring of the liquid, the amount of the liquid that flowed out from the bottom side of the cuff, that is, from the distal side, is measured. Then, the ratio of the amount of the liquid that flowed out from the bottom side of the cuff to the amount of the liquid poured from above the cuff, that is, the trickle ratio, is measured. According to its definition, the trickle ratio can be considered to be an index representing how easily the foreign matter such as accidentally-swallowed object that accumulated on the upstream side of the cuff flows or trickles to the downstream side of the cuff.

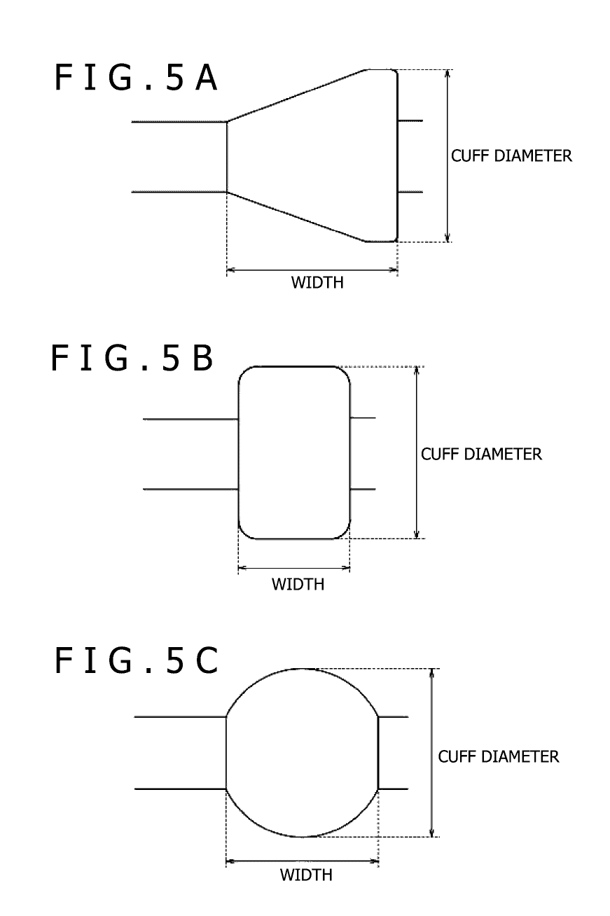

Next, three types of the shape of the cuff used in the experiments by the present inventors will be explained below. In the experiments, the present inventors classified the cuff shapes into the three types shown in FIGS. 5A to 5C. As shown in FIG. 5A, the first type is in a shape like a circular truncated cone with its upper base situated on the distal side of the tracheal tube and its lower base situated on the proximal side of the tracheal tube. In other words, the first type is in a tapered shape with the diameter of the outer surface increasing as it goes from the distal side to the proximal side. This type will hereinafter be referred to as a “tapered type.” Note that, even in cases like FIG. 5A where a part of the circular truncated cone on the lower base side is not tapered, the cuff is classified under the tapered type if the cuff has a tapered shape as a whole.

As shown in FIG. 5B, the second type is in a shape like a cylinder whose outer diameter is larger than the diameter of the tube main body. This type will hereinafter be referred to as a “tire type.” The trunk portion of a cuff of the tire type has a straight-line part that is in parallel with the tube main body in the lateral view.

As shown in FIG. 5C, the third type has an outer shape like a sphere. This type will hereinafter be referred to as a “spherical type.” The trunk portion of a cuff of the spherical type has curved surfaces in the lateral view.

The present inventors calculated the wrinkle area ratios and the trickle ratios by conducting the aforementioned first and second experiments by using a total of eight types of tracheal tubes, including three types of tracheal tubes having cuffs of the tapered type, two types of tracheal tubes having cuffs of the tire type, and three types of tracheal tubes having cuffs of the spherical type. As the wrinkle area ratio, the result of conducting the experiment twice under the same conditions is shown. Table 1 shows the result of the experiments conducted by the present inventors. In Table 1, the eight types of tracheal tubes with the cuffs are respectively represented as TUBE 1 to TUBE 8.

TABLE 1

| | TUBE 1 | TUBE 2 | TUBE 3 | TUBE 4 | TUBE 5 | TUBE 6 | TUBE 7 | TUBE 8 |
|---|---|---|---|---|---|---|---|---|
| | TAPERED | TAPERED | TAPERED | TIRE | TIRE | SPHERICAL | SPHERICAL | SPHERICAL |
| SHAPE | TYPE | TYPE | TYPE | TYPE | TYPE | TYPE | TYPE | TYPE |
| CUFF | 26 | 26 | 27 | 19 | 26 | 30 | 27 | 25 |
| DIAMETER | | | | | | | | |
| (mm) | | | | | | | | |
| AVERAGE | 0.06 | 0.06 | 0.14 | 0.06 | 0.08 | 0.08 | 0.11 | 0.23 |
| FILM | | | | | | | | |
| THICKNESS | | | | | | | | |
| (mm) | | | | | | | | |
| AVERAGE | 0.34 | 0.17 | 0.55 | 0.43 | 0.59 | 0.32 | 0.30 | 0.12 |
| YOUNGS | | | | | | | | |
| MODULUS | | | | | | | | |
| (MPa) | | | | | | | | |
| WRINKLE | 5.1 | 9.7 | 13.9 | 7.5 | 10.5 | 6.0 | 7.1 | 13.0 |
| AREA RATIO | | | | | | | | |
| (%) | | | | | | | | |
| TRICKLE | 0.65 | 13.2 | 94 | 3.5 | 53 | 18 | 70 | 80 |
| RATIO (%) | | | | | | | | |

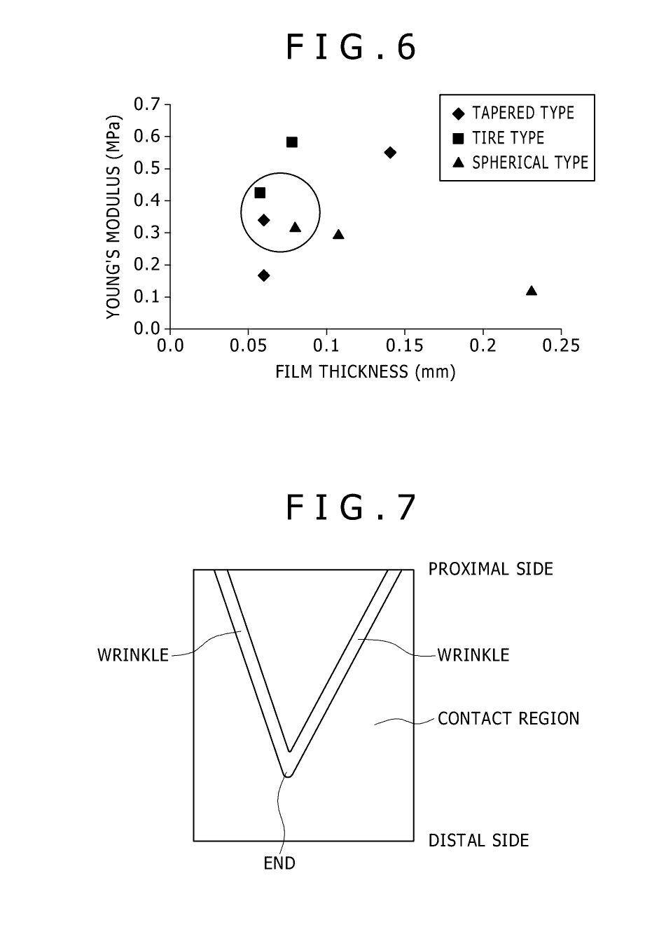

FIG. 6 is a chart made by plotting the eight types of tracheal tubes used in the experiments by the present inventors (i.e., TUBE 1 to TUBE 8) based on characteristics of the cuff. In the chart of FIG. 6, tracheal tubes with cuffs inside the region surrounded by a circle have the tendency that the wrinkle area ratio and the trickle ratio are low in comparison with tracheal tubes with cuffs outside the circular region. Thus, the present inventors, considering that the wrinkle area ratio and the trickle ratio tend to be low when the cuff satisfies conditions inside the region, conducted three confirmation experiments.

A first confirmation experiment is a confirmation experiment regarding the film thickness. The present inventors carried out the first confirmation experiment as follows: Cuffs differing from each other in the film thickness were made with the same material. By using tracheal tubes equipped with the cuffs made as above, the wrinkle area ratios and the trickle ratios were measured by use of the syringe simulating the trachea in the same way as the aforesaid first and second experiments.

Specifically, in the first confirmation experiment, a total of six types of cuffs, the tire type and the tapered type at film thicknesses of 0.06 mm, 0.15 mm and 0.25 mm, were made. In both of the tire type and the tapered type, the cuff diameter was set at 26 mm. In the tire type cuffs, the width was set at 15 mm. In the tapered type cuffs, the taper angle θ was set at 25°. For every cuff, flexible polyvinyl chloride A (Young's modulus: 0.34 MPa), made by adjusting the compounding ratios of the plasticizer and the stabilization agent for flexible polyvinyl chloride, was used as the cuff material. Table 2 shows the result of the first confirmation experiment conducted by the present inventors. As the wrinkle area ratio, the result of conducting the experiment twice under the same conditions is shown.

TABLE 2

| | FILM THICKNESS (mm) | | | | | |
|---|---|---|---|---|---|---|
| | 0.06 | | 0.15 | | 0.25 | |
| | SHAPE | | | | | |
| | TIRE | TAPERED | TIRE | TAPERED | TIRE | TAPERED |
| | TYPE | TYPE | TYPE | TYPE | TYPE | TYPE |
| WRINKLE AREA RATIO (%) | 14.8 | 5.1 | 31.9 | 31.8 | — | — |
| TRICKLE RATIO (%) | 6.3 | 0.65 | 100 | 92 | 65 | 100 |

As shown in Table 2, in both of the tire type and the tapered type, the cuff with 0.06 mm film thickness resulted in a low wrinkle area ratio, that is, unlikelihood of wrinkles formed on the outer surface of the cuff. This can be attributed to easiness of uniform stretch of the outer surface of the cuff by the pressure of the fluid supplied to the annular space in the inflation of the cuff with 0.06 mm film thickness in comparison with the cuffs with 0.15 mm and 0.25 mm film thicknesses. Note that the cuffs with 0.25 mm film thickness did not fully inflate, and thus the wrinkle area ratios of these cuffs could not be measured. The trickle ratio was also lower with the cuffs with 0.06 mm film thickness.

A second confirmation experiment is a confirmation experiment regarding the shape. The present inventors carried out the second confirmation experiment as follows: Cuffs differing from each other in the shape were made with the same material and at the same film thickness. By using tracheal tubes equipped with the cuffs made as above, the wrinkle area ratios were measured by use of the syringe simulating the trachea in the same way as the aforesaid experiments of the eight types of tracheal tubes.

In the second confirmation experiment, cuffs of the tire type, the tapered type and the spherical type were made. Tire type cuffs were made in three types: 10 mm wide, 15 mm wide and 20 mm wide. Tapered type cuffs were made in three types: taper angle θ=40°, 25° and 10°. A spherical type cuff was made to have a width (maximum diameter) of 20 mm. In all of the tire type, the tapered type and the spherical type, the cuff diameter was set at 26 mm, the cuff film thickness was set at 0.06 mm, and flexible polyvinyl chloride A (Young's modulus: 0.34 MPa) was used as the cuff material. Table 3 shows the result of the second confirmation experiment conducted by the present inventors. As the wrinkle area ratio, the result of conducting the experiment twice under the same conditions is shown.

TABLE 3

| | SHAPE | | | | | | |
|---|---|---|---|---|---|---|---|
| | | | | | | | SPHERICAL |
| | TIRE TYPE | | | TAPERED TYPE | | | TYPE |
| WIDTH (mm) | 12 | 17 | 22 | 15 | 19 | 33 | 20 |
| TAPER ANGLE | — | — | — | 40° | 25° | 10° | — |
| WRINKLE AREA RATIO (%) | 11.7 | 14.8 | 16.2 | 4.7 | 5.1 | 3.8 | 7.2 |
| TRICKLE RATIO (%) | 5.2 | 6.3 | 16.9 | 2.2 | 0.65 | 0 | 1.4 |

As shown in Table 3, in comparison with the cuffs of the tire type and the spherical type, the tapered type cuffs resulted in low wrinkle area ratios, that is, unlikelihood of wrinkles formed on the outer surface of the cuff. This can be attributed to the fact that in the inflation of a tapered type cuff, the proximal side of the tapered part having the larger diameter first contacts the inner wall of the trachea and the contact region of the cuff and the inner wall of the trachea gradually extends from the contacting part towards the distal side. The trickle ratio was also lower in the tapered type cuffs in comparison with the cuffs of the tire type and the spherical type.

A third confirmation experiment is a confirmation experiment regarding the material. The present inventors carried out the third confirmation experiment as follows: Cuffs of the same shape and film thickness were made with different materials. By using tracheal tubes equipped with the cuffs made as above, the wrinkle area ratios were measured by use of the syringe simulating the trachea in the same way as the aforesaid experiments of the eight types of tracheal tubes.

In the third confirmation experiment, tapered type cuffs having a film thickness of 0.06 mm were made with two types of materials. One cuff was made with the aforementioned flexible polyvinyl chloride A having a Young's modulus of 0.34 MPa. The other cuff was made with flexible polyvinyl chloride B having a Young's modulus of 0.17 MPa. The taper angle θ and the diameter of the cuffs were set at 25° and 26 mm, respectively. Table 4 shows the result of the third confirmation experiment conducted by the present inventors. As the wrinkle area ratio, the result of conducting the experiment twice under the same conditions is shown.

TABLE 4

| | FLEXIBLE | FLEXIBLE |
|---|---|---|
| | POLYVINYL | POLYVINYL |
| MATERIAL | CHLORIDE A | CHLORIDE B |
| YOUNGS MODULUS (MPa) | 0.34 | 0.17 |
| WRINKLE AREA RATIO (%) | 5.1 | 9.7 |
| TRICKLE RATIO (%) | 0.65 | 13.2 |

As shown in Table 4, in comparison with the cuff made with flexible polyvinyl chloride B, the cuff made with flexible polyvinyl chloride A resulted in a low wrinkle area ratio, that is, unlikelihood of wrinkles formed on the outer surface of the cuff. The trickle ratio was also lower in the cuff made with flexible polyvinyl chloride A in comparison with the cuff made with flexible polyvinyl chloride B.

Based on the result of the above-described experiments, the present inventors conducted the aforementioned first and second experiments by using the tracheal tube 1 equipped with the cuff 3 of this embodiment shown in FIG. 3 as a cuff having a shape suitable for reducing the wrinkle area ratio and the trickle ratio. Table 5 shows the result of the experiments of the tracheal tube 1 equipped with the cuff 3 of this embodiment. As the wrinkle area ratio, the result of conducting the experiment twice under the same conditions is shown.

TABLE 5

| | |
|---|---|
| WIDTH (mm) | 26 |
| TAPER ANGLE | 12° |
| DIAMETER (rim) | 26 |
| MATERIAL | FLEXIBLE |
| | POLYVINYL |
| | CHLORIDE A |
| YOUNGS MODULUS (MPa) | 0.34 |
| FILM THICKNESS (mm) | 0.06 |
| WRINKLE AREA RATIO (%) | 4.4 |
| TRICKLE RATIO (%) | 0 |

As shown in Table 5, the cuff 3 of this embodiment is capable of reducing the wrinkle area ratio. Accordingly, a gap due to wrinkles hardly appears between the outer surface of the cuff 3 and the inner wall of the patient's trachea, and a gap allowing in foreign matter such as accidentally-swallowed object hardly appears between the outer surface of the cuff 3 and the inner wall of the patient's trachea. Thus, with the cuff 3 of this embodiment, the amount of foreign matter escaping the suction by the first lumen decreases. By the cuff 3 and the tracheal tube 1 equipped with the cuff 3, the foreign matter suction performance can be improved.

The result shown in Table 5 will be examined further below. With the cuff 3 of this embodiment, the trickle ratio was 0%, that is, no liquid flowed out to the distal side of the tracheal tube 1 even when liquid was poured into the tracheal tube 1 from the proximal side. Thus, considering the fact that the wrinkle area ratio is not 0% (at least 1% or higher, for example), it can be understood that even though wrinkles form on the cuff 3, the wrinkles do not connect from the proximal side to the distal side on the cuff 3 and even wrinkles connecting to the proximal side or the distal side of the contact region do not connect to the other side (i.e., the distal side or the proximal side). Therefore, by the cuff 3 of this embodiment, the inflow of foreign matter such as accidentally-swallowed object into the lungs via a gap caused by wrinkles is inhibited. Accordingly, the onset risk of aspiration pneumonitis can be reduced by the cuff 3 of this embodiment.

The reason why the formation of wrinkles connecting from the proximal side to the distal side is inhibited by the cuff 3 of this embodiment can be understood as follows: For example, since the cuff 3 has a tapered shape, the circumference of an outer surface part formed by a cross section orthogonal to the central axis line O1 gradually narrows as it goes from the proximal side towards the distal side of the tracheal tube. Thus, as schematically shown in FIG. 7, a plurality of wrinkles (two wrinkles in FIG. 7) that is formed to connect to the proximal side of the tracheal tube come close to each other towards the distal side, are interlocked with each other at a position before reaching the distal side rather than each reaching the distal side and connecting from the proximal side to the distal side, and form a wrinkle end at the interlocked position. Thus, the wrinkles do not extend beyond the wrinkle end towards the distal side and the formation of wrinkles connecting from the proximal side to the distal side is inhibited.

Note that the present disclosure is not limited to the configurations specified in the aforementioned embodiment and a variety of modifications are possible within an extent not departing from the subject matter of the disclosure described in the claims. For example, while the distal portion 8, the cuff attachment portion 9 and the proximal portion 11 of the aforementioned tube main body 2 have been assumed to be non-curved, straight tubular parts, it is also possible to form the entire tube main body 2 from the distal portion to the proximal portion as a curved tube main body.

Further, the conditions of the cuff 3 may be selected properly based on the aforementioned experiments within a range in which the wrinkle area ratio becomes lower than a prescribed value. For example, while the Young's modulus of the material of the cuff 3 was specified as 0.34 in the aforesaid embodiment, it is also possible to use a cuff made with a material having a Young's modulus within a range greater than or equal to 0.32 MPa and less than or equal to 0.39 MPa, for example, for the tracheal tube 1. While the film thickness of the cuff 3 was specified as 0.06 mm in the aforesaid embodiment, it is also possible to use a cuff having a film thickness within a range greater than or equal to 0.06 mm and less than 0.15 mm, for example, for the tracheal tube 1. Preferably, the film thickness of the cuff 3 is greater than or equal to 0.06 mm and less than or equal to 0.10 mm. More preferably, the film thickness of the cuff 3 is greater than or equal to 0.06 mm and less than or equal to 0.08 mm. While the taper angle θ was specified as 12° in the aforesaid embodiment, it is also possible to use a cuff having a taper angle θ within a range greater than or equal to 10° and less than or equal to 12°, for example, for the tracheal tube 1.

Put another way, a wrinkle area ratio less than or equal to a prescribed threshold value less than or equal to 4.4%, such as a wrinkle area ratio less than or equal to 3%, can be realized by properly setting the film thickness of the trunk portion within the range greater than or equal to 0.06 mm and less than 0.15 mm, the Young's modulus of the film material within the range greater than or equal to 0.32 MPa and less than or equal to 0.39 MPa, and the taper angle θ within the range greater than or equal to 10° and less than or equal to 12°.

Furthermore, while the welding portions 14 and 15 in FIG. 3 showing the cuff 3 of the aforesaid embodiment are welded to the tube main body 2 so as to extend from the trunk portion 16 towards the distal side and the proximal side, the mode of the welding of the welding portions 14 and 15 is not limited to this example. For example, the welding of the cuff 3 to the tube main body 2 may also be carried out by folding back the welding portions 14 and 15 towards the trunk portion 16 and welding the welding portions 14 and 15 to the tube main body 2 inside the trunk portion 16. It is also possible to fold back only one of the welding portions 14 and 15 towards the trunk portion 16.

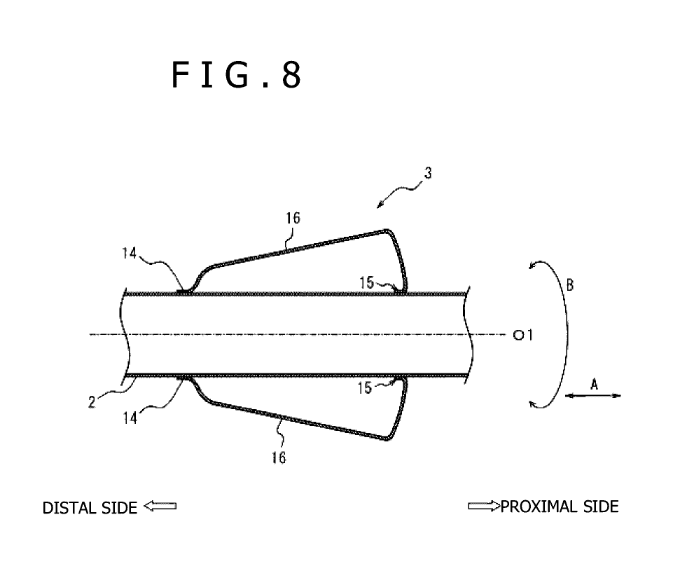

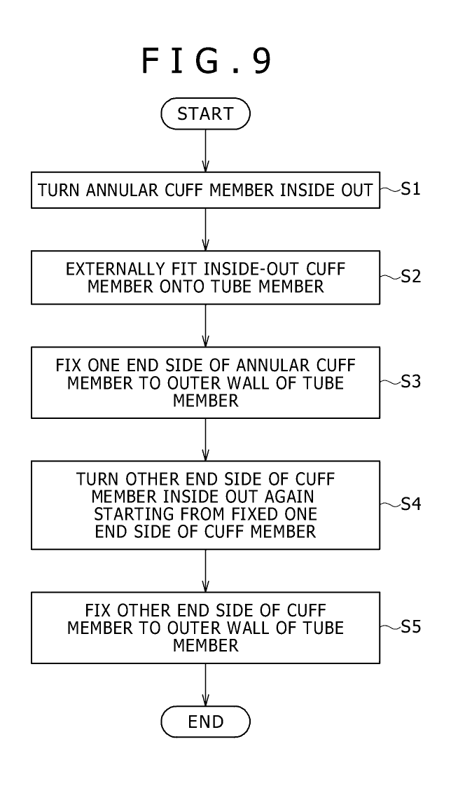

FIG. 8 is a diagram showing an example of a cross section of the cuff 3 with the welding portion 14 welded in the state of extending from the trunk portion 16 towards the distal side and the welding portion 15 welded in the state of being folded back towards the trunk portion 16. The method of attaching the cuff 3 shown in FIG. 8 will be explained below with reference to FIG. 9.

As shown in FIG. 9, the method of attaching the cuff 3 includes step S1 of turning an annular cuff member inside out, step S2 of inserting a tube member into the inside-out cuff member and externally fitting the inside-out cuff member onto the tube member, step S3 of fixing one end side of the annular cuff member to the outer wall of the tube member, step S4 of turning a part of the cuff member on the other end side inside out again starting from the fixed one end side of the cuff member, and step S5 of fixing the other end side of the cuff member to the outer wall of the tube member. Note that the “tube member” means the completed tube main body 2 or a member as the base of the tube main body 2. The “annular cuff member” means a member that is attached to the tube member and thereby constitutes the cuff 3 of the tracheal tube 1, that is, a member as the base of the cuff 3. The annular cuff member has a large-diameter part to become the trunk portion and end portions extending from both ends of the large-diameter part and having smaller diameters than the large-diameter part. The steps will be explained in detail below.

In step S1, the annular cuff member is turned inside out so that a surface of the large-diameter part to become the inner wall of the trunk portion is exposed to the outside. Incidentally, step S1 can be left out in cases where the annular cuff member has no such discrimination between the front and back sides or the annular cuff member has already been turned inside out.

In step S2, the inside-out annular cuff member is externally fitted onto the tube member from one end side of the tube member (e.g., one end side to become the proximal end 6 of the tube main body 2). It is also possible to externally fit the inside-out annular cuff member onto the tube member from the other end side of the tube member (e.g., one end side to become the distal end 5 of the tube main body 2).

In step S3, an end portion on one end side of the annular cuff member externally fitted onto the tube member, that is, an end portion to become the welding portion 15, is fixed to the outer wall of the tube member by means of welding or the like. Note that, when the end portion on the one end side of the cuff member is fixed to the outer wall of the tube member, the end portion on the one end side of the cuff member is in the state of extending to the outside of the large-diameter part in a central axis line direction of the tube member (in the same direction as the central axis line direction A of the tube main body 2). As for the end portion on the one end side to be fixed to the tube member and to become the welding portion 15, when the end portion on the one end side is fixed to the outer wall of the tube member, the cuff member is in the inside-out state and thus the end portion on the one end side is situated on the distal side of the tube member in comparison with the large-diameter part, that is, situated in a direction towards the distal end 5 of the tube main body 2 from the large-diameter part.

In step S4, the other end side of the cuff member is turned inside out again starting from the fixed end portion on the one end side of the cuff member. Specifically, the part of the cuff member on the other end side is turned inside out so that a surface of the large-diameter part to become the outer wall of the trunk portion is exposed to the outside. More specifically, an end portion on the other end side of the annular cuff member, that is, an end portion to become the welding portion 14, and the large-diameter part to become the trunk portion are turned inside out starting from the welding portion 15. As a result, the welding portion 15 takes on a state of being folded back towards the large-diameter part to become the trunk portion in the central axis line direction of the tube member. On the other hand, the end portion to become the welding portion 14 takes on a state of extending to the outside of the large-diameter part to become the trunk portion (specifically, a state of extending from the large-diameter part towards the distal side of the tube member) in the central axis line direction of the tube member.

In step S5, the end portion on the other end side of the cuff member, as the end portion to become the welding portion 14, is fixed to the outer wall of the tube member by means of welding or the like, by which the fixation of the cuff member to the tube member is completed.

The cuff 3 can be attached to the tube member by the method described above. While the method of attaching the cuff 3 has been described here with reference to FIG. 9, for the cuff 3 shown in FIG. 3, it is possible to execute a step of externally fitting an annular cuff member to become the cuff 3 onto the tube member without turning the annular cuff member inside out and then fixing one end side of the annular cuff member to the outer wall of the tube member and a step of fixing the other end side of the cuff member to the outer wall of the tube member. More specifically, the welding portions 14 and 15 are formed by fixing both end portions of the annular cuff member externally fitted onto the tube member, the both end portions extending to the outside of the large-diameter part to become the trunk portion in the directions of the central axis line O1 of the tube member, to the outer wall of the tube member by means of welding or the like.

The present disclosure relates to a cuff and a tracheal tube.

Having described the preferred embodiment of the present disclosure with reference to the accompanying drawings, it is to be understood that the disclosure is not limited to the precise embodiment and that various changes and modifications could be effected therein by one skilled in the art without departing from the spirit or scope of the disclosure as defined by the appended claims.

What is claimed is:

1. A cuff having a trunk portion capable of deflation and inflation, wherein
the trunk portion in a state of the inflation is rotationally symmetric with respect to a prescribed axis and has a tapered part whose diameter gradually increases centering at the prescribed axis as it goes from one end side towards the other end side of the prescribed axis, an angle made by the prescribed axis and the tapered part is greater than or equal to 10° and less than or equal to 12°, and
a wrinkle area ratio as a ratio between a wrinkle area of a part of the trunk portion where wrinkles form and a contact area of a part of the trunk portion contacting an inner wall of a trachea in a state in which the trunk portion is inflated in the trachea is less than or equal to 4.4%.

2. The cuff according to claim 1, wherein a film thickness of the trunk portion is greater than or equal to 0.06 mm and less than 0.15 mm.

3. The cuff according to claim 1, wherein the trunk portion is formed with a material having a Young's modulus greater than or equal to 0.32 MPa and less than or equal to 0.39 MPa.

4. A tracheal tube comprising
a tube main body equipped with the cuff according to claim 1, wherein
the prescribed axis is a central axis line of an outer peripheral surface of the tube main body, and
the one end side is a distal side of the tube main body.
